(19) United States
(12) Patent Application Publication
(10) Pub. No.: US 2024/0216462 A1
(43) Pub. Date: Jul. 4, 2024

(54) PHARMACEUTICAL COMPOSITION FOR ENHANCING IMMUNITY OR ANTI-CANCER ACTIVITY INCLUDING EXTRACT OF PSEUDOLYSIMACHION NAKAIANUM (OHWI) T.YAMAZ AS ACTIVE INGREDIENT

(71) Applicant: KOREA INSTITUTE OF SCIENCE AND TECHNOLOGY, Seoul (KR)

(72) Inventors: Jaeyoung KWON, Gangneung-si (KR); Hak Cheol Kwon, Gangneung-si (KR); Won Kyu Kim, Gangneung-si (KR); Choong-Gu Lee, Gangneung-si (KR); InWha Park, Gangneung-si (KR); Hyeon-Seong Lee, Gangneung-si (KR); Sungmin Song, Gangneung-si (KR); Hoseong Hwang, Gangneung-si (KR); Soon Kwang Lee, Gangneung-si (KR); Jong Gwon Baek, Gangneung-si (KR); Dong Hoon Kim, Gangneung-si (KR); Yujin Kwon, Gangneung-si (KR); Su-Yeon Cho, Gangneung-si (KR); Dae Sik Jang, Gangneung-si (KR)

(21) Appl. No.: 18/530,106

(22) Filed: Dec. 5, 2023

(30) Foreign Application Priority Data
Dec. 21, 2022 (KR) 10-2022-0180733

(51) Int. Cl. A61K 36/68; A61K 31/7034; A61K 31/7048

(52) U.S. Cl. CPC A61K 36/68; A61K 31/7034; A61K 31/7048; A61K 2236/333; A61K 2236/39

(57) ABSTRACT

The present disclosure is completed by confirming the differentiation into Th17 and its activity-increasing function and the differentiation into Treg and its activity-suppressing function in compounds isolated from Pseudolysimachion nakaianum (Ohwi) T. Yamaz. The composition of the present disclosure increases the immune response by simultaneously regulating Th17 and Treg cell groups and their activities, and thus may be applied for the prevention and treatment of cancer and infectious diseases. According to the present disclosure, there is an advantage of having very few or no side effects by using Pseudolysimachion nakaianum (Ohwi) T. Yamaz, a natural product with safety that has been verified for a long time. Therefore, the present disclosure is expected to be used in various fields such as drugs, quasi-drugs, cosmetic materials, functional biomaterials, and functional food materials for immunity reinforcement and improvement, prevention, or treatment of infectious diseases and tumors.

### CROSS-REFERENCE TO RELATED APPLICATION

This application claims the benefit of Korean Patent Application No. 10-2022-0180733 filed on Dec. 21, 2022, in the Korean Intellectual Property Office, the entire disclosure of which is incorporated herein by reference for all purposes.

### BACKGROUND

### 1. Field of the Invention

The present disclosure relates to use for prevention or treatment of diseases (e.g., cancer) requiring immunity reinforcement or immunity reinforcement of an extract of Pseudolysimachion nakaianum (Ohwi) T. Yamaz and an iridoid derivative and a phenylpropanoid derivative derived from the extract, which has been completed by confirming differentiation into Th17 and an activity-increasing function thereof, and differentiation into Treg and an activity-inhibiting function thereof in an iridoid derivative and a phenylpropanoid derivative, which are compounds isolated from Pseudolysimachion nakaianum (Ohwi) T. Yamaz.

### 2. Description of the Related Art

CD4+ T cells consist of various subgroups according to their phenotypes and functional properties. The CD4+ T cells that produce IL-17 cytokine are classified as Th17 and play an important role in the elimination of foreign pathogens and fungi. Regulatory T cells (Treg) are a subgroup that serves to maintain homeostasis and self-tolerance of an immune system by suppressing an immune response, and plays an important role to inhibit cell proliferation and cytokine production and prevent autoimmunity.

Cell differentiation and functional development of Th17 and Treg are closely associated with each other, and the balance between the two cell groups is essential for immune homeostasis. If there is a problem in maintaining the balance of Th17 and Treg cell groups, various inflammatory reactions such as autoimmunity, allograft rejection, and tumor formation are caused.

The Treg cells are important for maintaining immune homeostasis, but during tumor development, the Treg cells accumulate locally in tumors and induce tumor immune evasion by inducing anergy and immune suppression of the cells. Specific inhibition and inactivation of the Treg cells may cause tumor-specific T cell activation and improve the effectiveness of cancer immunotherapy.

In addition, the Treg cells play an important role in suppressing an immunopathogenic response to infection. In chronic infection, when the function and generation of the Treg cells are suppressed, the infection control ability is improved by increasing the immune response mediated by CD8+ T cells. In the case of acute infection, the Treg cells are also a main cause of inhibition of pathogen elimination ability through suppression of a cytotoxic lymphocyte (CTL) response. On the contrary, an increase in Th17 has been reported to have positive functions such as elimination of infectious pathogens and enhancement of tumor elimination. Attempts have been made to find methods and regulatory candidate substances for reducing Treg or increasing Th17, but reports on compounds that simultaneously control the balance of the two subgroup cells are extremely rare.

The present inventors have completed the present disclosure by intensively researching to develop a natural product-derived substance capable of helping in preventing and treating tumors and infectious diseases through a method of simultaneously controlling the decrease and malfunction of Treg and the increase and function improvement of Th17.

### SUMMARY

An aspect of the present disclosure is to provide a composition for enhancing immunity or anti-cancer activity derived from natural products.

However, technical aspects of the present disclosure are not limited to the aforementioned purpose and other aspects which are not mentioned may be clearly understood to one of ordinary skill in the art from the following description.

According to an aspect, there is provided a composition for enhancing immunity or anti-cancer activity including an extract of Pseudolysimachion nakaianum (Ohwi) T. Yamaz as an active ingredient.

According to another aspect, there is provided a food composition for enhancing immunity or anti-cancer activity including an extract of Pseudolysimachion nakaianum (Ohwi) T. Yamaz as an active ingredient.

According to another aspect, there is provided a method of enhancing immunity or anti-cancer activity including administering an extract of Pseudolysimachion nakaianum (Ohwi) T. Yamaz to a subject. The method may further include measuring the expression level of IL17 or Foxp3 in a biological sample isolated from a subject before and after administration of the extract of Pseudolysimachion nakaianum (Ohwi) T. Yamaz.

According to another aspect, there is provided a use of an extract of Pseudolysimachion nakaianum (Ohwi) T. Yamaz for preparing a drug for enhancing immunity or anti-cancer activity.

In an embodiment of the present disclosure, the extract of Pseudolysimachion nakaianum (Ohwi) T. Yamaz may be a whole extract of Pseudolysimachion nakaianum (Ohwi) T. Yamaz, and may be extracted by using ethanol, desirably 70% ethanol as an extraction solvent.

In another embodiment of the present disclosure, the extract of Pseudolysimachion nakaianum (Ohwi) T. Yamaz may be a butanol fraction of an ethanol extract of Pseudolysimachion nakaianum (Ohwi) T. Yamaz.

In another embodiment of the present disclosure, the extract of Pseudolysimachion nakaianum (Ohwi) T. Yamaz may promote a differentiation of CD4+ T cells into Th17 cells and suppress a differentiation of CD4+ T cells into regulatory T cells.

In another embodiment of the present disclosure, the extract of Pseudolysimachion nakaianum (Ohwi) T. Yamaz may include a compound 1 represented by Chemical Formula 1 below, a compound 2 represented by Chemical Formula 2 below, and/or a compound 3 represented by Chemical Formula 3 below.

In another embodiment of the present disclosure, the extract of Pseudolysimachion nakaianum (Ohwi) T. Yamaz may include derivatives of a compound 1 represented by Chemical Formula 1 below, a compound 2 represented by Chemical Formula 2 below, and/or a compound 3 represented by Chemical Formula 3 below, stereoisomers thereof, pharmaceutically acceptable salts thereof, hydrates thereof, or solvates thereof.

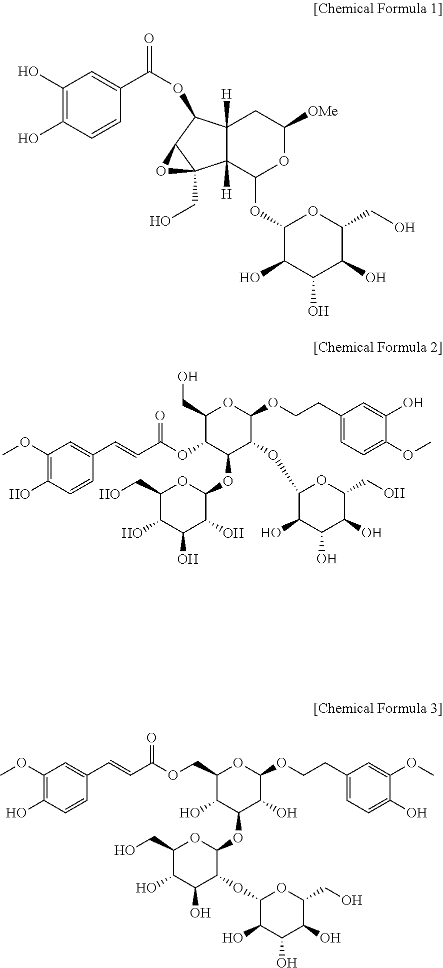

According to another aspect, there is provided a composition for enhancing immunity or anti-cancer activity including at least one compound selected from the compound 1 represented by Chemical Formula 1 above, the compound 2 represented by Chemical Formula 2 above, and the compound 3 represented by Chemical Formula 3 above.

According to another aspect, there is provided a food composition for enhancing immunity or anti-cancer activity including at least one compound selected from the compound 1 represented by Chemical Formula 1 above, the compound 2 represented by Chemical Formula 2 above, and the compound 3 represented by Chemical Formula 3 above as an active ingredient.

According to another aspect, there is provided a method of enhancing immunity or anti-cancer activity including administering at least one compound selected from the compound 1 represented by Chemical Formula 1 above, the compound 2 represented by Chemical Formula 2 above, and the compound 3 represented by Chemical Formula 3 above to a subject. The method may further include measuring the expression level of IL17 or Foxp3 in a biological sample isolated from a subject before and after administration of the compound 1, the compound 2, and/or the compound 3.

According to another aspect, there is provided a use of the compound 1, the compound 2, and/or the compound 3 for enhancing immunity or anti-cancer activity.

According to another aspect, there is provided a method of extracting the compound 1, the compound 2, and/or the compound 3 including the following steps:
(1) obtaining an extract of Pseudolysimachion nakaianum (Ohwi) T. Yamaz by extracting Pseudolysimachion nakaianum (Ohwi) T. Yamaz leaves with ethanol;
(2) fractionating the extract of Pseudolysimachion nakaianum (Ohwi) T. Yamaz with water, hexane, butanol, ethyl acetate, and butanol sequentially; and
(3) performing a column chromatography with the butanol fraction.

Additional aspects of embodiments will be set forth in part in the description which follows and, in part, will be apparent from the description, or may be learned by practice of the disclosure.

According to embodiments, there is an advantage of having very few or no side effects by using a compound isolated and identified from Pseudolysimachion nakaianum (Ohwi) T. Yamaz, a natural product with safety that has been verified for a long time. Further, the compound of the present disclosure may effectively promote an immune response by simultaneously having an effect of differentiation into Th17 and enhancement of its functions, and an effect of differentiation into Treg and suppression of its functions, and is expected to be used in various fields such as drugs for preventing or treating diseases requiring activation of immune response such as cancer and infectious diseases, quasi-drugs, cosmetic materials, functional biomaterials, and functional food materials.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a schematic diagram of a process for isolating compounds 1 to 3 from an extract of Pseudolysimachion nakaianum (Ohwi) T. Yamaz;

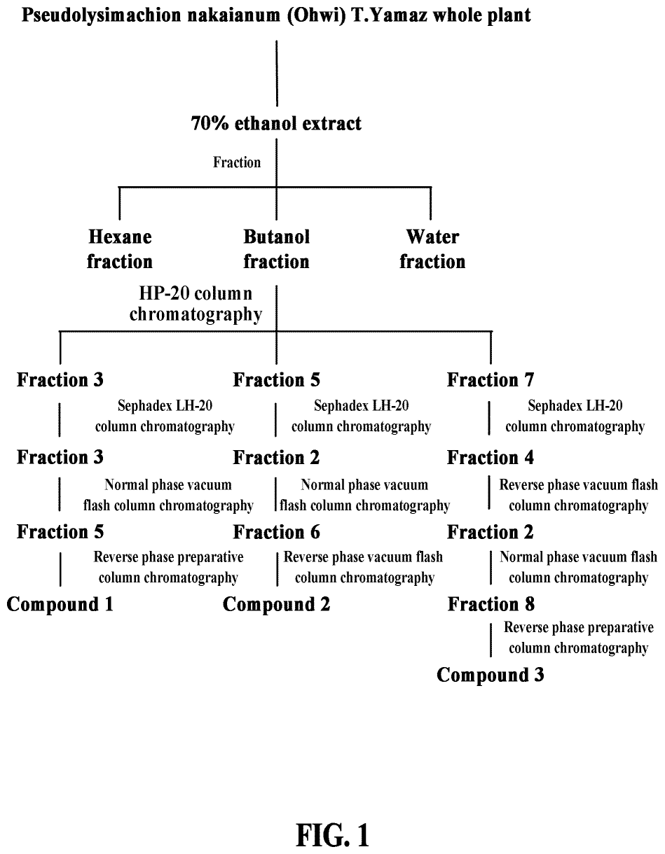

FIG. 2 is a chromatogram of a high performance liquid chromatography (HPLC) analysis of an ethanol extract of Pseudolysimachion nakaianum (Ohwi) T. Yamaz;

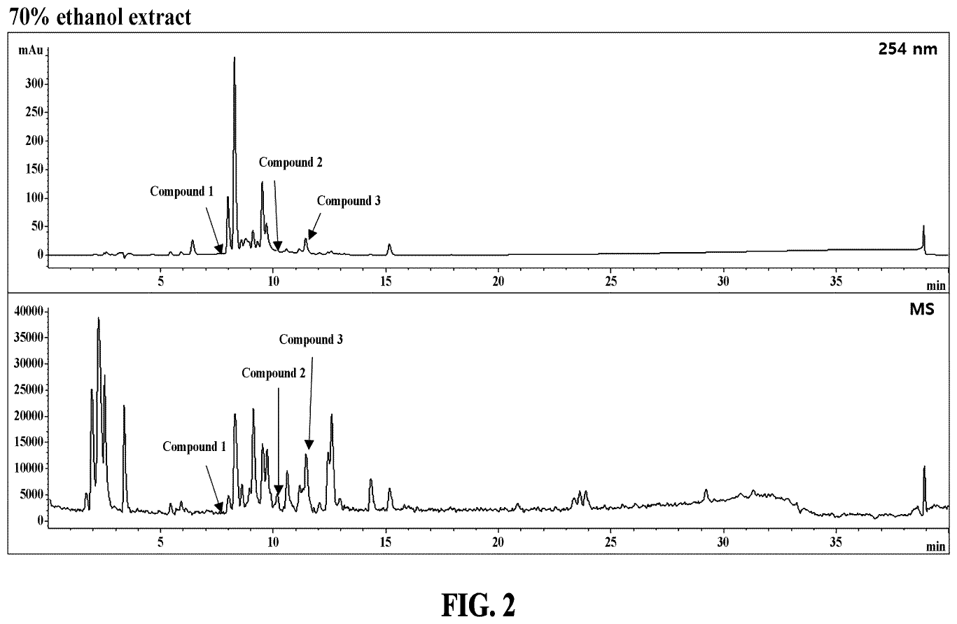

FIG. 3 is a chromatogram of an HPLC analysis of a butanol fraction of the ethanol extract of Pseudolysimachion nakaianum (Ohwi) T. Yamaz;

FIG. 4 is a diagram for confirming the degree of Th17 differentiation and its activity according to the treatment of compounds 1 to 3 in CD4+ T cells induced to differentiate into Th17 through the expression level of IL-17; and

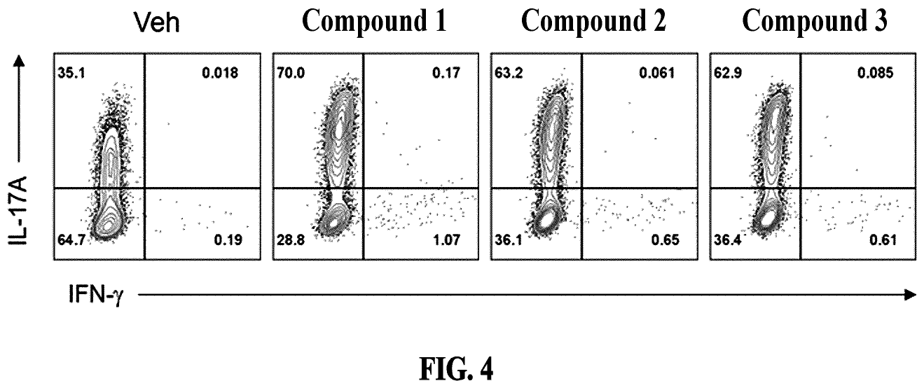

FIG. 5 is a diagram for confirming the degree of Treg differentiation and its activity according to the treatment of compounds 1 to 3 in CD4+ T cells induced to differentiate into Treg through the expression level of Foxp3.

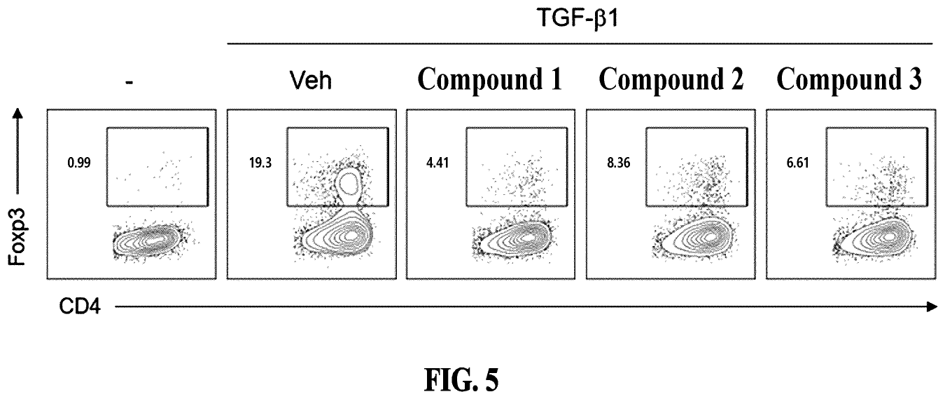

### DETAILED DESCRIPTION

Pseudolysimachion nakaianum (Ohwi) T. Yamaz is a perennial plant belonging to the Scrophulariaceae, and little research has been made on the ingredients of Pseudolysimachion nakaianum (Ohwi) T. Yamaz and applications thereof.

The present inventors isolated compounds 1 to 3 represented by Chemical Formulas 1 to 3 below from an extract of Pseudolysimachion nakaianum (Ohwi) T. Yamaz.

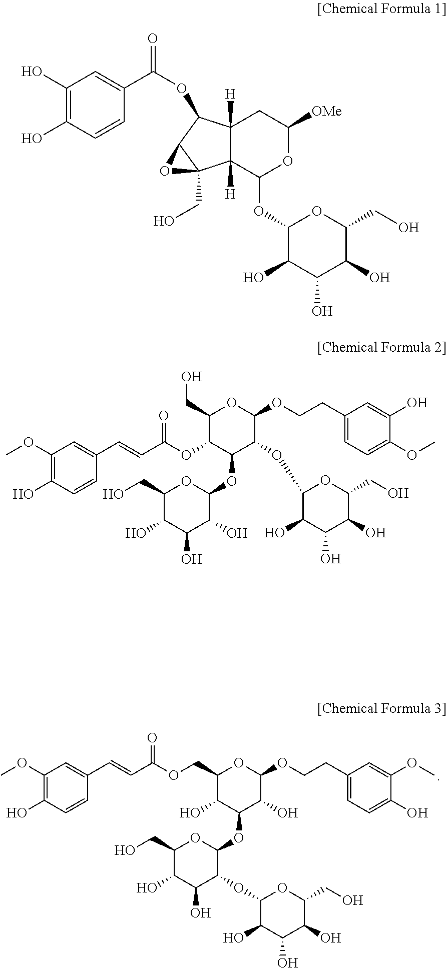

As a result of NMR spectrum analysis using a nuclear magnetic resonance spectrometer, the compounds 1 and 3 were identified as novel compounds, and the present inventors named the compounds as veroninakaioside G and veroninakaioside H, respectively.

In addition, the present inventors confirmed through a specific experiment that all of the compounds 1 to 3 stimulated differentiation into Th17 of CD4+ T cells, which had induced the differentiation into Th17, and increased the secretion of IL-17, and confirmed that all of the compounds 1 to 3 promoted the differentiation into Treg of CD4+ T cells, which had induced differentiation into regulatory T cells, so that it was determined that the compounds 1 to 3 may be used to increase the immune response.

Accordingly, an aspect of the present disclosure is to provide a composition for immunity reinforcement including an extract of Pseudolysimachion nakaianum (Ohwi) T. Yamaz as an active ingredient. The composition of the present disclosure may be used as a pharmaceutical composition for immunity reinforcement, and may be used as a food composition for immunity reinforcement.

In addition, the present disclosure provides a use of at least one compound selected from the group consisting of the compounds 1 to 3 for immunity reinforcement. The at least one compound selected from the group consisting of the compounds 1 to 3 may be provided in a pharmaceutical composition for immunity reinforcement, and may be provided in a food composition for immunity reinforcement.

Meanwhile, the extract of Pseudolysimachion nakaianum (Ohwi) T. Yamaz of the present disclosure and compounds 1 to 3 derived therefrom have the activity of promoting the immune response, and thus may be used for tumor immunotherapy.

Accordingly, the present disclosure provides a composition for preventing, improving, or treating cancer including an extract of Pseudolysimachion nakaianum (Ohwi) T. Yamaz or at least one compound selected from the group consisting of the compounds 1 to 3 as an active ingredient. The composition may be provided as a pharmaceutical composition for preventing or treating cancer, and may be provided as a food composition for preventing or improving cancer.

In the present disclosure, the extract of Pseudolysimachion nakaianum (Ohwi) T. Yamaz may be extracted with any solvent that has been previously used, desirably may be extracted with a solvent selected from the group consisting of water, lower alcohols having 1 to 4 carbon atoms, polyhydric alcohols having 3 to 6 carbon atoms, glycerin, propylene glycol, butylene glycol, and mixed solvents thereof, but may be extracted as a solvent, desirably ethanol, more desirably 70% ethanol.

The extraction method using the extraction solvent may use conventional extraction methods in the art, for example, a stationary extraction method, an ultrasonic extraction method, a subcritical extraction method, a high temperature extraction method, a high pressure extraction method, a filtration method, or a reflux extraction method, desirably a stationary extraction method, but is not limited thereto.

In addition, the extract of Pseudolysimachion nakaianum (Ohwi) T. Yamaz may be additionally fractionated by a conventional systematic fractionation method. Specifically, the Pseudolysimachion nakaianum (Ohwi) T. Yamaz fraction may be a hexane fraction, a butanol fraction, and a water fraction obtained by suspending the extract of Pseudolysimachion nakaianum (Ohwi) T. Yamaz in distilled water, sequentially fractionating the extract using hexane and butanol, and obtaining the remaining distilled water soluble, but is not limited thereto.

In the present disclosure, the extract of Pseudolysimachion nakaianum (Ohwi) T. Yamaz may also be used as it is, but may be used in the form of a concentrated extract after filtration or in the form of a lyophilized product after freeze-drying.

The present inventors obtained an extract of Pseudolysimachion nakaianum (Ohwi) T. Yamaz using 70% ethanol, fractionated the extract of Pseudolysimachion nakaianum (Ohwi) T. Yamaz with hexane and butanol, and then isolated and identified the compound 1 (veroninakaioside G) represented by Chemical Formula 1, the compound 2 (chionoside I) represented by Chemical Formula 2, and the compound 3 (veroninakaioside H) represented by Chemical Formula 3.

When the butanol fraction of Pseudolysimachion nakaianum (Ohwi) T. Yamaz was eluted with an appropriate solvent from methanol/water of 0:100 to methanol/water of 100:0 using silica HP-20 column chromatography and divided into 10 sub-fractions, the compound 1 was isolated from the third sub-fraction, the compound 2 was isolated from the fifth sub-fraction, and the compound 3 was isolated from the seventh sub-fraction.

In the present disclosure, the compounds 1 to 3 may be used in the form of pharmaceutically acceptable salts, and may be provided as stereoisomers thereof, hydrates thereof, or solvates thereof.

As used herein, the term “pharmaceutically acceptable salt” means a salt in a form that may be used pharmaceutically among salts as materials in which cations and anions are bonded by electrostatic attraction, and may be generally a metal salt, a salt with an organic base, a salt with an inorganic acid, a salt with an organic acid, a salt with a basic or acidic amino acid, and the like. For example, the metal salt may be an alkali metal salt (sodium salt, potassium salt, etc.), an alkaline earth metal salt (calcium salt, magnesium salt, barium salt, etc.), an aluminum salt, and the like. The salt with the organic base may be salts with triethylamine, pyridine, picoline, 2,6-lutidine, ethanolamine, diethanolamine, triethanolamine, cyclohexylamine, dicyclohexylamine, N,N-dibenzylethylenediamine, etc. The salt with the inorganic acid may be salts with hydrochloric acid, hydrobromic acid, nitric acid, sulfuric acid, phosphoric acid, etc. The salt with the organic acid may be salts with formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid, and the like. The salt with the basic amino acid may be salts with arginine, lysine, ornithine, and the like, and the salt with the acidic amino acid may be salts with aspartic acid, glutamic acid, and the like. When the compound has an acidic functional group therein, a particularly desirable salt may be inorganic salts such as alkali metal salts (e.g., sodium salt, potassium salt, etc.), alkaline earth metal salts (e.g., calcium salt, magnesium salt, barium salt, etc.); and organic salts such as an ammonium salt. When the compound has a basic functional group therein, the particularly desirable salt may be salts with inorganic acids such as hydrochloric acid, hydrobromic acid, nitric acid, sulfuric acid, phosphoric acid, etc., and salts with organic acids such as acetic acid, phthalic acid, fumaric acid, oxalic acid, tartaric acid, maleic acid, citric acid, succinic acid, methanesulfonic acid, p-toluenesulfonic acid, etc.

The composition of the present disclosure may further include at least one known active ingredient having an effect of immunity reinforcement or prevention or treatment of cancer, in addition to the extract of Pseudolysimachion nakaianum (Ohwi) T. Yamaz or at least one compound selected from the group consisting of the compounds 1 to 3.

The composition of the present disclosure may further include suitable carriers, excipients, and diluents which are commonly used in the preparation of pharmaceutical composition. In addition, the composition may be formulated and used in the form of oral formulations, such as powders, granules, tablets, capsules, suspensions, emulsions, syrups, aerosols, etc., external preparations, suppositories, and sterile injectable solutions according to a general method.

The carrier, the excipient, and the diluent that may be included in the composition may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, etc. In the case of formulating the composition, the formulations may be prepared by using a diluent or an excipient, such as a filler, an extender, a binder, a wetting agent, a disintegrating agent, and a surfactant, which are generally used.

Solid formulations for oral administration include tablets, pills, powders, granules, capsules, etc., and these solid formulations may be prepared by mixing at least one excipient, for example, starch, calcium carbonate, sucrose, lactose, gelatin, and the like with the composition. Further, lubricants such as magnesium stearate and talc are used in addition to simple excipients. Liquid formulations for oral administration may correspond to suspensions, oral liquids, emulsions, syrups, and the like, and may include various excipients, for example, a wetting agent, a sweetener, an aromatic agent, a preserving agent, and the like, in addition to water and liquid paraffin which are commonly used as simple diluents. Chemical Formulations for parenteral administration include sterilized aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used.

In addition, the present disclosure provides a method for immunity reinforcement and a method for preventing or treating cancer, including administering the composition to a subject, and a use of the composition for preparing drugs or anticancer drugs for immunity reinforcement.

The “subject” used herein refers to a subject having a disease requiring an increase in immune response or having a high probability of developing the disease, and more particularly, refers to mammals such as human or non-human primates, mice, rats, dogs, cats, horses and cattle. In addition, the disease requiring the increase in the immune response includes diseases caused by infections of viruses, bacteria, and fungi, and cancer.

The term “administration” used herein means providing a predetermined composition of the present disclosure to a subject by any suitable method.

A desirable dose of the pharmaceutical composition of the present disclosure varies according to the condition and body weight of a patient, the degree of a disease, a drug form, and the route and period of administration, but may be properly selected by those skilled in the art. For the desired effect, the extract of Pseudolysimachion nakaianum (Ohwi) T. Yamaz of the present disclosure, a fraction thereof, or the compound 1 and/or 2 derived therefrom may be administered in an amount of 0.1 mg/kg to 1000 mg/kg a day, desirably 0.001 to 200 mg/kg, and may also be administered once or several times a day.

The pharmaceutical composition of the present disclosure may be administered to a subject through various routes. All methods of administration may be expected and for example, the pharmaceutical composition may be administered by oral, rectal or intravenous, intramuscular, subcutaneous, and topical injection.

The composition of the present disclosure may be added to food for the purpose of immunity reinforcement, but the food may be a health functional food, and in the present disclosure, the health functional food refers to food having a bioregulatory function such as prevention or improvement of a disease, biological defense, immunity, and recovery after illness, and should be harmless to the human body when taken for a long time.

When the composition of the present disclosure is used as a food additive, the extract of Pseudolysimachion nakaianum (Ohwi) T. Yamaz or the compound 1, the compound 2, and/or the compound 3 may be added as it is or used with other foods or food ingredients, and may be appropriately used according to a conventional method. The mixed amount of the active ingredients may be suitably determined according to a purpose of use (prevention, health, or therapeutic treatment). In general, when preparing foods or beverages, the composition of the present disclosure is added in an amount of 15 wt % or less, desirably 10 wt % or less, based on the raw material. However, in the case of long-term intake for the purpose of health and hygiene or for the purpose of health control, the amount of the composition may be equal to or greater than the range, and there is no problem in terms of safety, so that the active ingredients may be used even in an amount above the range.

The kind of food is not particularly limited. Examples of the food which may be added with the materials include meat, sausage, bread, chocolate, candies, snacks, confectionery, pizza, ramen, other noodles, gums, dairy products including ice cream, various soups, beverages, tea, drinks, alcohol drinks, vitamin complexes, and the like, and include all health foods in an accepted meaning.

The health beverage composition of the present disclosure, like a general beverage, may include various flavoring agents or natural carbohydrates as an additional ingredient. As the above-mentioned natural carbohydrates, monosaccharides such as glucose and fructose, disaccharides such as maltose and sucrose, natural sweeteners such as dextrin and cyclodextrin, synthetic sweeteners such as saccharin and aspartame, and the like may be used. A ratio of the natural carbohydrates may be generally about 0.01 to 10 g, desirably about 0.01 to 0.1 g per 100 ml of the composition of the present disclosure.

In addition to the ingredients, the composition of the present disclosure may include various nutrients, vitamins, electrolytes, flavoring agents, coloring agents, pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, or the like. In addition, the composition of the present disclosure may include pulps for preparing natural fruit juices, fruit juice beverages or vegetable beverages. These ingredients may be used independently or in combination. Although the ratio of these additives is not greatly important, generally, the ratio thereof is selected in a range of 0.01 to 0.1 part by weight per 100 parts by weight of the composition of the present disclosure.

Hereinafter, embodiments will be described in detail with reference to the accompanying drawings. However, since various modifications may be made to embodiments, the scope of the present disclosure is not limited or restricted by these embodiments. It should be understood that all modifications, equivalents and substitutes for embodiments are included in the scope of the present disclosure.

The terms used in embodiments are used for the purpose of description only, and should not be construed to be limited. The singular expression includes the plural expression unless the context clearly dictates otherwise. In the present disclosure, it should be understood that term “comprising” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art, and are not interpreted as ideal or excessively formal meanings unless otherwise defined in the present disclosure.

In addition, in the description with reference to the accompanying drawings, like components designate like reference numerals regardless of reference numerals and a duplicated description thereof will be omitted. In describing the embodiments, a detailed description of related known technologies will be omitted if it is determined that they unnecessarily make the gist of the embodiments unclear.

### EXAMPLES

### Example 1: Preparation of Extract of Pseudolysimachion Nakaianum (Ohwi) T. Yamaz and Fractions Thereof

Pseudolysimachion nakaianum (Ohwi) T. Yamaz whole plant was obtained in June 2020 at Hantaek Botanical Garden in Yongin, Gyeonggi-do. 2.1 kg of the whole plant was dried with warm air for 24 hours or more to obtain a dry weight of 747.5 g. The dried Pseudolysimachion nakaianum (Ohwi) T. Yamaz whole plant was sliced with a crusher, added with 70% ethanol/water (7.5 L), shaken and stirred at room temperature, left for 7 days, and extracted. The mixture was subjected to gravity filtration repeated twice using Whatman paper filter paper having a film thickness of 0.34 mm and a glass funnel having a diameter of 30 cm (twice ‘extraction-filtration’) to obtain a filtered extract. The filtered extract was put into a vacuum concentrator and completely evaporated and concentrated under reduced pressure to obtain 127.3 g of an extract of Pseudolysimachion nakaianum (Ohwi) T. Yamaz (yield: 17%).

The obtained crude extract was dissolved in water (540 mL), and then contacted with hexane (540 mL) three times to obtain a hexane layer and the hexane layer was distilled under reduced pressure to obtain 4.1 g of a hexane fraction (yield: 3.2%). After removing the hexane layer, the remaining water layer was contacted with butanol (540 mL) three times to obtain a butanol layer and the butanol layer was distilled under reduced pressure to obtain 46.1 g of a butanol fraction (yield: 36.2%). Finally, the remaining water layer was distilled under reduced pressure to obtain 64.9 g of a water fraction (yield: 51.0%), which was lyophilized and then stored.

### Example 2: Isolation and Structure Identification of Compounds 1 to 3 from Extract of Pseudolysimachion Nakaianum (Ohwi) T. Yamaz

### 2-1) Isolation of Compounds 1 to 3

Isolation of Compound 1: A butanol fraction was eluted with an appropriate solvent from methanol/water of 0:100 to methanol/water of 100:0 using HP-20 column chromatography (6.0×44.0 cm column), and divided into a total of 10 sub-fractions. The third sub-fraction (3.9 g) was eluted with methanol/water 50:50 using LH-20 column chromatography (4.7×64.0 cm column) and divided into 9 sub-fractions. The third sub-fraction (545.7 mg) was eluted with an appropriate solvent from dichloromethane/methanol/water of 90:9:1 to dichloromethane/methanol/water of 60:36:4 using normal phase vacuum flash column chromatography (silica 80 g) and then divided into 9 sub-fractions. The fifth sub-fraction (48.6 mg) was subjected to reverse phase preparative column chromatography under conditions shown in Table 1 below to isolate 8.6 mg of Compound 1 having the structure of Chemical Formula 1.

TABLE 1

| | |
|---|---|
| Use equipment | Waters 996 HPLC |
| Column | Gemini NX-C18 RP HPLC column |
| | (20.0 × 250 mm, 5 μm) |
| Solvent | (a) water (b) acetonitrile |
| Mobile phase | Elution for 80 minutes with (a) solvent:(b) |
| composition | solvent at volume ratio of 75:15 at 0 min |
| Mobile phase | 7 mL/min |
| flow rate | |
| Detector | UV 254 nm |

Isolation of Compound 2: The fifth sub-fraction (2.5 g) among 10 sub-fractions divided using HP-20 column chromatography was eluted with methanol/water 60:40 using LH-20 column chromatography (4.7×64.0 cm column) and divided into 10 sub-fractions. The second sub-fraction (240.8 mg) was eluted with an appropriate solvent from dichloromethane/methanol/water of 95:4.5:0.5 to dichloromethane/methanol/water of 70:27:3 using normal phase vacuum flash column chromatography (silica 40 g) and then divided into 8 sub-fractions. The sixth sub-fraction (50.7 mg) was subjected to reverse phase preparative column chromatography under conditions shown in Table 2 below to isolate 18.6 mg of Compound 2 having the structure of Chemical Formula 2.

TABLE 2

| | |
|---|---|
| Use equipment | Waters 996 HPLC |
| Column | Gemini NX-C18 RP HPLC column |
| | (20.0 × 250 mm, 5 μm) |
| Solvent | (a) water (b) acetonitrile |
| Mobile phase | Elution started with (a) solvent:(b) |
| composition | solvent at volume ratio 78:22 at 0 min |
| | and then (b) solvent ratio increased from |
| | 22% to 30% from 0 min to 80 min |
| Mobile phase | 7 mL/min |
| flow rate | |
| Detector | UV 254 nm |

Isolation of Compound 3: The seventh sub-fraction (2.5 g) among 10 sub-fractions divided using HP-20 column chromatography was eluted with methanol/water 60:40 using LH-20 column chromatography (4.7×64.5 cm column) and divided into 14 sub-fractions. The fourth sub-fraction was eluted with an appropriate solvent from methanol/water 40:60 to methanol/water 60:40 using reverse phase vacuum flash chromatography (C18 130 g), and divided into 5 sub-fractions. The second sub-fraction (1.0 g) was eluted with an appropriate solvent from dichloromethane/methanol/water of 100:0:0 to dichloromethane/methanol/water of 70:27:3 using normal phase column chromatography (2.9×29.5 cm column) and then divided into 12 sub-fractions. The eighth sub-fraction (54.6 mg) was subjected to reverse phase preparative column chromatography under conditions shown in Table 3 below to isolate 17.5 mg of Compound 3 having the structure of Chemical Formula 3.

TABLE 3

| | |
|---|---|
| Use equipment | Waters 996 HPLC |
| Column | LUNA C18 RP HPLC column |
| | (30.0 × 250 mm, 10 μm) |
| Solvent | (a) water (b) acetonitrile |
| Mobile phase | Elution for 80 minutes with (a) solvent:(b) |
| composition | solvent at volume ratio of 55:45 at 0 min |
| Mobile phase | 7 mL/min |
| flow rate | |
| Detector | UV 254 nm |

### 2-2) Identification of Compound 1

The molecular weight of Compound 1 isolated in Example 2-1 was determined as 530 through MS measurement using a high-resolution mass spectrometer, and through 1H and 13C NMR spectrum analysis using a nuclear magnetic resonance (NMR 500 Hz, JNM-ECZ500R), the structure was identified as a novel compound having the structure of Chemical Formula 1 below, which was called veroninakaioside G.

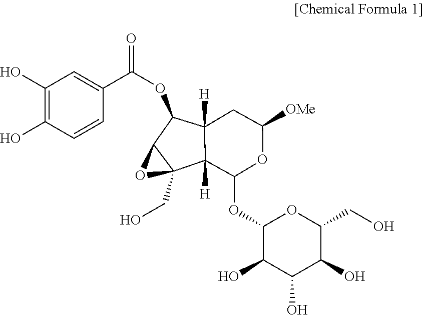 [SMILES: CO[C@@H]1C[C@H]2[C@H](OC(=O)c3ccc(O)c(O)c3)[C@@H]3O[C@]3(CO)[C@H]2C(O[C@@H]2O[C@H](CO)[C@@H](O)[C@H](O)[C@H]2O)O1]

Molecular Chemical Formula C₂₃H₃₀O₁₄; Light yellow powder; HR-Q-TOF-MS (positive mode) m/z=531.1703 [M+H]⁺; calculated for C₂₃H₃₁O₁₄, 539.1714); [a]_{D}²⁰:−158.0° (c 0.1, MeOH); UV (MeOH) λₘₐₓ 262 (3.73), 294 (3.57); IR (FT-IR) vₘₐₓ 3395, 2926, 1705, 1604, 1445, 1204, 1219, 1079 cm−1

¹H-NMR (CD₃OD, 500 MHz): δH 7.46 (1H, d, J=2.0 Hz, H-5″), 7.46 (1H, dd, J=8.5, 2.0 Hz, H-6″), 6.82 (1H, d, J=9.0 Hz, H-2″), 5.34 (1H, dd, J=9.0, 1.5 Hz, H-6), 5.23 (1H, d, J=7.5 Hz, H-1), 4.77 (1H, d, J=8.0 Hz, H-1′), 4.76 (1H, dd, J=12.0, 3.0 Hz, H-3), 4.11 (1H, d, J=13.0 Hz, H-10a), 3.94 (1H, dd, J=12.0, 2.0 Hz, H-6a), 3.79 (1H, d, J=13.0 Hz, H-10b), 3.72 (1H, d, J=1.5 Hz, H-7), 3.65 (1H, dd, J=12.0, 7.0 Hz), 3.40 (1H, t, J=9.0 Hz, H-3′), 3.50 (3H, s, H—OCH3), 3.34 (1H, m, H-4′), 3.26 (2H, m, H-2′, 5′), 2.49 (1H, t, J=7.5 Hz, H-9), 2.45 (1H, m, H-5), 1.84 (1H, dt, J=14.5, 3.0 Hz, H-4a), 1.67 (1H, ddd, J=15.0, 9.0, 6.0 Hz, H-4b)

¹³C-NMR (CD₃OD, 125 MHZ): 8C 168.5 (C-7″), 152.3 (C-3″), 146.5 (C-4″), 124.1 (C-6″), 122.3 (C-1″), 117.7 (C-2″), 116.1 (C-5″), 100.4 (C-2), 99.6 (C-1′), 95.6 (C-1), 79.0 (C-5′), 78.1 (C-3′), 77.6 (C-6), 75.1 (C-2′), 72.0 (C-4′), 66.7 (C-8), 63.2 (C-6′), 61.2 (C-10), 60.2 (C-7), 56.8 (C—OCH3), 43.4 (C-9), 36.3 (C-5), 29.7 (C-3, 4)

### 2-3) Identification of Compound 2

The molecular weight of Compound 2 isolated in Example 2-1 was determined as 830 through MS measurement using a mass spectrometer, and through 1H and 13C NMR spectrum analysis using a nuclear magnetic resonance (NMR 500 Hz, JNM-ECZ500R), the structure was estimated as chionoside I having the structure of Chemical Formula 2 below. In addition, the structure was identified by comparing the NMR data with data of the existing literature (Taskova et al. Chem. Pharm. Bull, 2010, 58, 703-711).

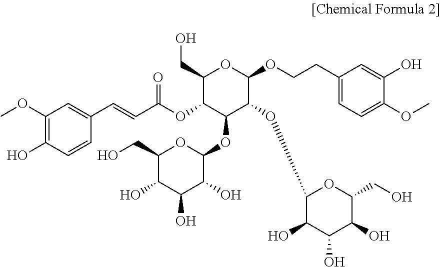

Molecular Chemical Formula C₃₇H₅₀O₂₁; ESI-MS: m/z 848 [M+NH4]⁺;

¹H-NMR (CD₃OD, 500 MHz): δH 7.63 (1H, d, J=16.0 Hz, H-7″″), 7.25 (1H, d, J=2.0 Hz, H-2″″), 7.10 (1H, dd, J=8.0, 2.0 Hz, H-6), 6.84 (1H, d, J=8.0 Hz, H-5″″), 6.81 (1H, d, J=8.0 Hz, H-5), 6.79 (1H, d, J=2.0 Hz, H-2), 6.72 (1H, dd, J=8.0, 2.0 Hz, H-6), 6.46 (1H, d, J=16.0 Hz, H-8″″), 4.93 (1H, overlapped, H-1′), 4.68 (1H, d, J=8.0 Hz, H-1″), 4.64 (1H, d, J=8.0 Hz, H-1″), 4.54 (1H, d, J=8.0 Hz, H-1′), 4.11 (2H, m, H-3′, 8a), 3.90 (3H, s, OCH3-3″), 3.82 (3H, s, OCH3-4), 3.80 (1H, m, H-6′″a), 3.78 (1H, m, H-6″a), 3.74 (1H, m, H-8b, 2′), 3.67 (2H, m, H-6′b, 6′″b), 3.56 (2H, m, H-5′, 6′b), 3.44 (4H, m, H-3″, 4″, 6″, 3′″), 3.24 (3H, m, H-2″, 5″, 2′″), 3.12 (1H, dd, J=9.0, 7.5 Hz, H-5″′), 3.00 (1H, dd, J=10.0, 9.0 Hz, H-4′″), 2.82 (2H, m, H-7)

### 2-4) Identification of Compound 3

The molecular weight of Compound 3 isolated in Example 2-1 was determined as 830 through MS measurement using a high-resolution mass spectrometer, and through 1H and 13C NMR spectrum analysis using a nuclear magnetic resonance (NMR 500 Hz, JNM-ECZ500R), the structure was identified as a novel compound having the structure of Chemical Formula 3 below, which was called veroninakaioside H.

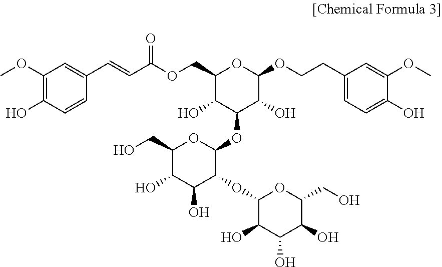

Molecular Chemical Formula C₃₇H₅₀O₂₁; Light yellow powder; HR-Q-TOF-MS (positive mode) m/z=831.2906 [M+H]⁺; calculated for C₃₇H₅₁O₂₁, 831.2923); [a]_{D}²⁰:−42.9° (c 0.1, MeOH); UV (MeOH) λₘₐₓ 262 (4.07), 288 (4.01), 326 (4.12); IR (FT-IR) vₘₐₓ 3412, 2921, 1512, 1270, 1162, 1031 cm⁻¹

¹H-NMR (CD₃OD, 500 MHz): δH 7.62 (1H, d, J=16.0 Hz, H-7″″), 7.15 (1H, d, J=2.0 Hz, H-2″″), 7.02 (1H, dd, J=8.0, 2.0 Hz, H-6″″), 6.80 (1H, d, J=8.0 Hz, H-5″″), 6.71 (1H, d, J=2.0 Hz, H-2), 6.66 (1H, d, J=8.0 Hz, H-5), 6.61 (1H, dd, J=8.0, 2.0 Hz, H-6), 6.38 (1H, d, J=16.0 Hz, H-8″″), 4.63 (1H, d, J=8.0 Hz, H-1″), 4.62 (1H, d, J=7.5 Hz, H-1′″), 4.52 (1H, dd, J=11.5, 2.0 Hz, H-6′a), 4.45 (1H, d, J=7.5 Hz, H-1′), 4.37 (1H, dd, J=11.5, 6.0 Hz, H-6′b), 3.91 (3H, m, H-8a, 6″a, 6″″a), 3.86 (3H, s, OCH3-3″″), 3.74 (3H, s, OCH3-4), 3.71 (2H, m, H-8b, 6′″b), 3.65 (1H, m, H-6″b), 3.61 (2H, m, H-5, 3′″), 3.50 (4H, m, H-2′, 3′, 4′, 2″), 3.36 (5H, m, H-4″, 5″, 3″, 4′″, 5″″), 3.28 (1H, dd, J=9.0, 7.5 Hz, H-2″′), 2.80 (1H, t, J=7.5 Hz, H-7)

¹³C-NMR (CD₃OD, 125 MHZ): 8C 169.2 (C-9″″), 150.8 (C-3″), 149.5 (C-4″″), 147.6 (C-3), 147.4 (C-4), 147.3 (C-7″″), 132.8 (C-1), 127.8 (C-1″″), 124.5 (C-6″″), 121.4 (C-6), 117.3 (C-2), 116.7 (C-5″″), 115.5 (C-8″), 112.9 (C-5), 111.8 (C-2″), 106.7 (C-1″), 104.2 (C-1″), 103.7 (C-1′), 90.2 (C-3′), 85.3 (C-2″), 79.0 (C-5″), 78.1 (C-5″), 77.7 (C-3″, 3″), 76.4 (C-2′″), 75.1 (C-5′), 74.0 (C-2′), 72.4 (C-8), 71.4 (C-4′″), 71.1 (C-4″), 70.5 (C-4′), 64.8 (C-6′), 62.5 (C-6″, 6′″), 55.1 (OCH3-4, 3″″), 36.9 (C-7)

### Example 3: Confirmation of Physiological Activity of Compounds 1 to 3

### 3-1) Confirmation of Differentiation and Functional Enhancement Activity of Th17 Cells

8-week-old C57BL/6J mice were purchased and adapted for 1 week, and then CD4+ T cells were isolated from spleens and lymph nodes. Th17 cell differentiation was induced by media containing IL-6, TGF-beta1, IL-1beta, anti-IFN-gamma, and anti-IL-4, and through culturing for 3 days, it was confirmed that about 35% of IL-17 cytokine was expressed in a Veh group, which was a control group treated with only a solvent of the compound, thereby establishing basic conditions for the differentiation of Th17 cells. Under the conditions of differentiation of Th17 cells, each substance was treated at a final concentration of 25 μM.

As a result, as may be seen in the drawings, it was confirmed that Compounds 1 to 3 increased the IL-17 cytokine expression by 70%, 63%, and 62%, respectively, and through this, it was confirmed that the three compounds were able to improve the differentiation and function of Th17 cells.

### 3-2) Confirmation of Differentiation and Functional Enhancement Activity of Treg Cells

8-week-old C57BL/6J mice were purchased and adapted for 1 week, and then CD4+ T cells were isolated from spleens and lymph nodes. Differentiation of regulatory T cells was transferred through a medium containing IL-2 and TGF-beta1, and through culture for 3 days, it was confirmed that about 19% of a Foxp3 transcription factor was expressed in the control group. Each substance was treated at a final concentration of 25 μM.

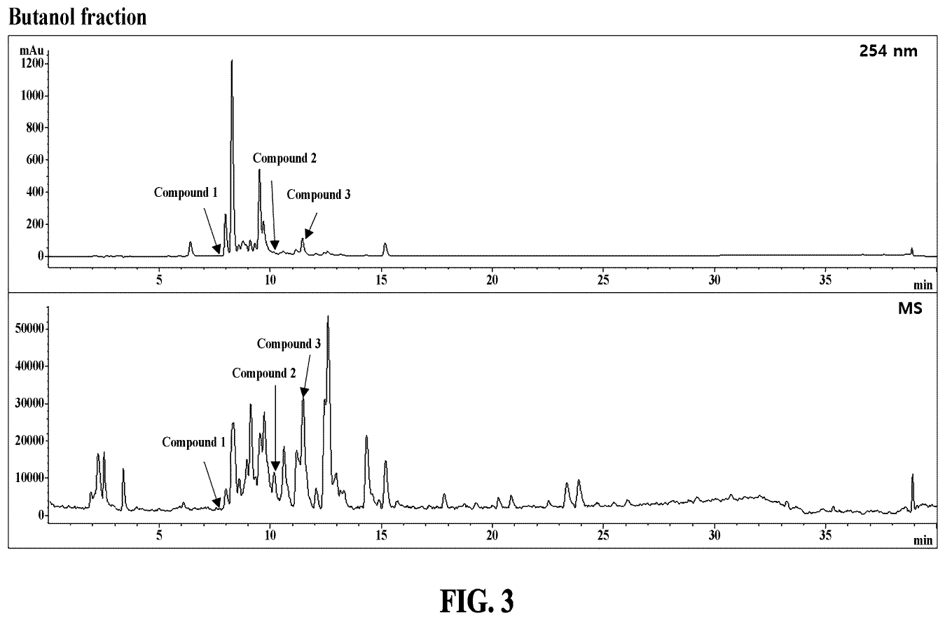

As a result, as illustrated in FIG. 3, it was confirmed that Compounds 1 to 3 reduced the Foxp3 transcription factor expression by 4.4%, 8.3%, and 6.6%, respectively, and through this, it was confirmed that the three compounds were able to inhibit or reduce the differentiation and function of regulatory T cells.

The aforementioned description of the present disclosure is used for exemplification, and it may be understood by those skilled in the art that the present disclosure may be easily modified in other detailed forms without changing the technical spirit or requisite features of the present disclosure. Therefore, it should be appreciated that the embodiments described above are illustrative in all aspects and are not restricted.

What is claimed is:

1. A method for enhancing immunity or anti-cancer activity comprising administering an extract of Pseudolysimachion nakaianum (Ohwi) T. Yamaz. to a subject.

2. The method of claim 1, wherein the extract is extracted with 70% ethanol as a solvent.

3. The method of claim 2, wherein the extract is a butanol fraction of an ethanol extract of Pseudolysimachion nakaianum (Ohwi) T. Yamaz.

4. The method of claim 1, wherein the extract of Pseudolysimachion nakaianum (Ohwi) T. Yamaz. comprises at least one compound selected from the group consisting of a compound 1 represented by Chemical Formula 1 below, a compound 2 represented by Chemical Formula 2 below, and a compound 3 represented by Chemical Formula 3 below.

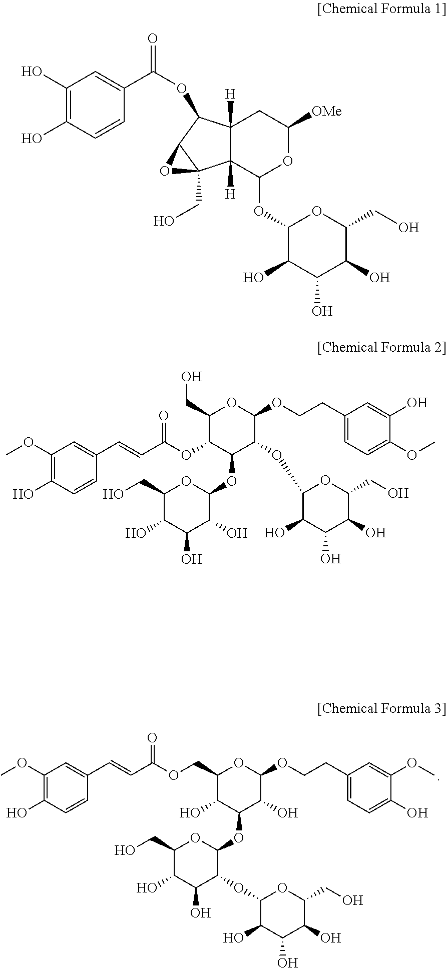

5. The method of claim 1, wherein the extract promotes a differentiation of CD4+ T cells into Th17 cells and suppresses a differentiation into regulatory T cells.

6. A method for enhancing immunity or anti-cancer activity comprising administering
at least one compound selected from the group consisting of a compound 1 represented by Chemical Formula 1 below, a compound 2 represented by Chemical Formula 2 below, and a compound 3 represented by Chemical Formula 3 below to a subject;
a derivative of the compound;
a stereoisomer of the compound;
a pharmaceutically acceptable salt of the compound;
a hydrate of the compound; or
a solvate of the compound.

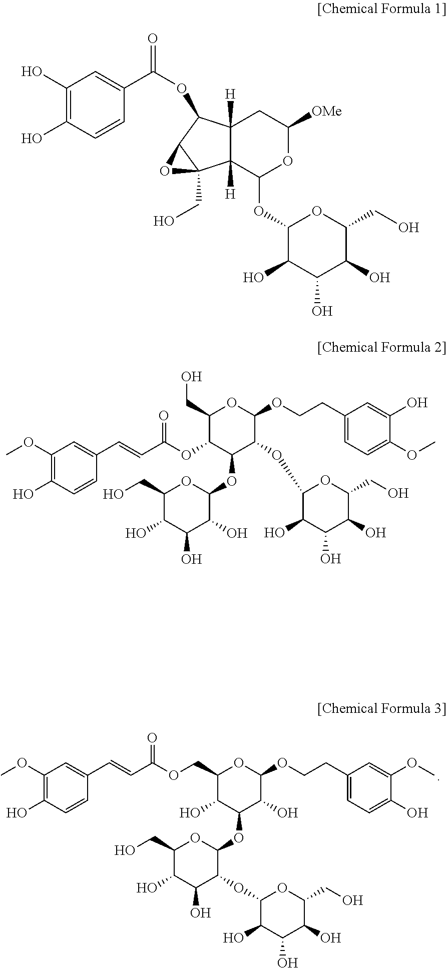

7. The method of claim 5, wherein the compound 1, the compound 2, and the compound 3 are derived from Pseudolysimachion nakaianum (Ohwi) T. Yamaz.

8. A method of extracting a compound 1 represented by Chemical Formula 1 below, a compound 2 represented by Chemical Formula 2 below, and a compound 3 represented by Chemical Formula 3 below, the method comprising:
(1) obtaining an extract of Pseudolysimachion nakaianum (Ohwi) T. Yamaz. Pseudolysimachion nakaianum (Ohwi) T. Yamaz. by extracting a Pseudolysimachion nakaianum (Ohwi) T. Yamaz. whole plant with ethanol;
(2) fractionating the extract of Pseudolysimachion nakaianum (Ohwi) T. Yamaz. by sequentially contacting the extract of Pseudolysimachion nakaianum (Ohwi) T. Yamaz. and each of water, hexane and butanol; and
(3) performing a column chromatography with the butanol fraction. [chemical structure]
